(19) United States
(12) Patent Application Publication
(10) Pub. No.: US 2001/0018405 A1
(43) Pub. Date: Aug. 30, 2001

(54) Process for preparation of pyrimidinone derivatives

(76) Inventor: Harald Walter, Rodersdorf (CH)

Correspondence Address: SYNGENTA, 3054 CORNWALLIS ROAD, RESEARCH TRIANGLE PARK, NC, 27709, US

(21) Appl. No.: 09/741,694

(22) Filed: Dec. 19, 2000

Related U.S. Application Data
(63) Continuation of application No. PCT/EP99/04188, filed on Jun. 17, 1999.

(30) Foreign Application Priority Data
Jun. 19, 1998 (GB) 9813238.4
Jun. 30, 1998 (GB) 9814088.2

(51) Int. Cl. C07D 239/72; C07D 239/70

(52) U.S. Cl. 504/235; 544/236; 544/279; 544/285

(57) ABSTRACT

A process for the preparation of compounds having plant protecting, in particular fungicidal properties, of the formula I

[chemical structure]

wherein A is a fused 5-membered heterocyclic ring which may be saturated or unsaturated, aromatic or non-aromatic and which may contain one or two hetero atoms O, S and/or N, or is fused benzo, pyrido or pyridazino; R₁ and R₂ are groups which are inert to the reactions; R₃ is optionally substituted C₁-C₈alkyl, C₂-C₈alkenyl, C₂-C₈alkynyl, C₃-C₆cycloalkyl or C₃-C₆ cycloalkyl-C₁-C₆alkyl; and R₄ is optionally substituted C₁-C₆alkyl, C₂-C₆alkenyl, C₂-C₆alkynyl, C₃-C₆cycloalkyl or C₃-C₆ cycloalkyl-C₁-C₆alkyl; in which process (a) a compound of the formula II, wherein A, R₁, R₂ and R₃ are as defined for formula I, is reacted with an orthocarbonate of the formula III, wherein R₄ is as defined for formula I and Y is OR₄, CN or NO₂, to give the intermediate compound of formula IV; and subsequently (b) the compound of the formula IV is cyclized to a compound of the formula I

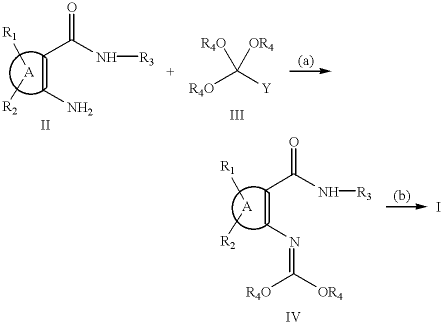

Process for preparation of pyrimidinone derivatives

The invention relates to a process for the preparation of compounds of formula I

[chemical structure]

wherein

A is a fused 5-membered heterocyclic ring which may be saturated or unsaturated, aromatic or non-aromatic and which may contain one or two hetero atoms O, S and/or N, or is fused benzo, pyrido or pyridazino;

R₁ and R₂ are groups which are inert to the reactions;

R₃ is C₁-C₈alkyl, C₂-C₈alkenyl, C₂-C₈alkynyl, C₃-C₆cycloalkyl or C₃-C₆cycloalkyl-C₁-C₆alkyl, each of which is unsubstituted or substituted by halogen; or is O—C₁-C₄alkyl, O—C₁-C₄haloalkyl, C₁-C₄alkoxy, S—C₁-C₄alkyl, SO—C₁-C₄alkyl, SO₂-C₁-C₄alkyl, CO—C₁C₄alkyl, N═CH—C₁-C₄alkyl, N═C(C₁-C₄alkyl)₂, NH—C₁-C₄alkyl, N(C₁-C₄alkyl)₂, COO—C₁-C₄alkyl, COO-aryl, cyano, nitro, Si—(C₁-C₄alkyl)₃, phenyl, halophenyl, phenoxyphenyl, halophenoxyphenyl or naphthyl; and R₄ is C₁-C₆alkyl, C₂-C₆alkenyl, C₂-C₆alkynyl, C₃-C₆cycloalkyl or C₃-C₆cycloalkyl-C₁-C₆alkyl, each of which is unsubstituted or substituted by halogen; or is O—C₁-C₄alkyl, O—C₁-C₄haloalkyl, C₁-C₄alkoxy, S—C₁-C₄alkyl, SO—C₁-C₄alkyl, SO₂-C₁-C₄alkyl, CO—C₁-C₄alkyl, N═CH—C₁-C₄alkyl, N═C(C₁-C₄alkyl)₂, NH—C₁-C₄alkyl, N(C₁-C₄alkyl)₂, COO—C₁-C₄alkyl, COO-aryl, cyano, nitro, Si—(C₁-C₄alkyl)₃, phenyl, halophenyl, phenoxyphenyl, halophenoxyphenyl or naphthyl; in which process

(a) a compound of the formula II, wherein A, R₁, R₂ and R₃ are as defined for formula I, is reacted with an orthocarbonate of the formula III, wherein R₄ is as defined for formula I and Y is OR₄, CN or NO₂, to give the intermediate compound of formula IV; and subsequently

(b) the compound of the formula IV is cyclized to a compound of the formula I.

[chemical structure]

Compounds of formula I having plant protecting, in particular fungicidal properties are known e.g. from WO 97/48684, WO 97/33890, WO 97/02262 and WO 94/26722. In the syntheses of these compounds as described therein thiophosgene or an isothiocyanate is used for the preparation of the the pyrimidinone moiety; in an additional subsequent reaction step, the sulfur introduced with the above reagents has to be eliminated.

The known processes for the preparation of compounds of formula I are accordingly unsatisfactory for economic and ecological reasons.

The method provided herewith is distinguished by good technical feasibility and is economically and ecologically more favorable.

Compounds of formula II are known e.g. from WO 97/33890.

Orthocarbonates of formula III are known or can be prepared by known methods, e.g. according to Liebigs Ann. Chem. 1982, 507-529.

Reaction step (a) is carried out with or without a solvent; the temperature is not critical and may vary from 20° to 200° C.; preferably is a temperature of 80° to 170° C., most preferably at or near the boiling temperature of the solvent.

The reaction is advantageously carried out in the presence of catalytic amounts of an acid. e.g 1-20% or 1-5% per weight, and in the absence of water. Suitable acids are mineral acids, typically sulfuric acid, phosphoric acid or a hydrogen halide, as HCl, HBr, HF; organic carboxylic acids, typically acetic acid, trifluoroacetic acid, oxalic acid, or organic sulfonic acids, typically methanesulfonic acid or p-toluenesulfonic acid.

Reaction step (b) is carried with or without a solvent; the temperature is not critical and may vary from 0° to 200° C.; preferably is a temperature of 30° to 150° C., most preferably at or near the boiling temperature of the solvent.

The reaction is advantageously carried out in the presence of a base, preferably in about equimolar amounts, as for example, alkali metal hydroxide or alkaline earth metal hydroxide, alkali metal hydride or alkaline earth metal hydride, alkali metal amide or alkaline earth metal amide, alkali metal alkanolate or alkaline earth metal alkanolate, alkali metal carbonate or alkaline earth metal carbonate, alkali metal dialkylamide or alkaline earth metal dialkylamide, or alkali metal alkylsilylamide or alkaline earth metal alkylsilylamide, alkylamines, alkylenediamines, optionally N-alkylated, optionally unsaturated cycloalkylamines, basic heterocycles, ammonium hydroxides and carbocyclic amines. Examples meriting mention are sodium hydroxide, sodium hydride, sodium amide, sodium methanolate, sodium carbonate, potassium tert-butanolate, potassium carbonate, lithium diisopropylamide, potassium bis(trimethylsilyl)amide, calcium hydride, triethylamine, triethylenediamine, cyclo-hexylamine, N-cyclohexyl-N,N-dimethylamine, N,N-diethylaniline, pyridine, 4-(N,N-dimethyl-amino)pyridine, N-methylmorpholine, benzyltrimethylammonium hydroxide, and 1,8-diaza-bicyclo[5.4.0]undec-5-ene (DBU).

Preferred are sodium hydride, potassium hydride, sodium amide, sodium methanolate, sodium carbonate, potassium tert-butanolate, potassium carbonate, lithium diisopropyl-amide, sodium hydroxide and potassium hydroxide.

Suitable solvents or diluents for both reaction steps (a) and (b) are for example: aromatic, aliphatic and alicyclic hydrocarbons and halogenated hydrocarbons, typically benzene, toluene, xylene, chlorobenzene, bromobenzene, petroleum ether, hexane, cyclohexane, dichloromethane, trichloromethane, dichloroethane or trichloroethane; ethers, typically diethyl ether, tert-butylmethyl ether, glyme, diglyme, tetrahydrofuran or dioxane; ketones, typically acetone or methyl ethyl ketone; alcohols, typically methanol, ethanol, propanol, butanol, ethylene glycol or glycerol; esters, typically ethyl acetate or butyl acetate; amides, typically N,N-dimethylformamide, N,N-dimethylacetamide, N-methylpyrrolidone or hexamethylphosphoric acid triamide; nitriles, typically acetonitrile; and sulfoxides, typically dimethylsulfoxide. Bases used in excess, such as triethylamine, pyridine, N-methylmorpholine or N,N-diethylaniline, can also be used as solvents or diluents in reaction step (b).

In a particular preferred mode the intermediate compound of formula IV is not isolated; according to this procedure compounds of formulae II and III are mixed and reacted together, optionally in presence of a solvent and of an acid as described above, until the reaction is completed.

The substituents R₁ and/or R₂ in ring A may be introduced or interchanged in the compounds of formula I as described in WO 97/33890.

In a particular embodiment of the present invention, R₁ and/or R₂ in ring A may be introduced or interchanged also on the intermediate compounds of formula IV.

This is particularly advantageous for the preparation of compounds of formula I, wherein R₁ and/or R₂ are halogen. In this process, a compound of the formula IV, in which R₁ and/or R₂ are hydrogen, is halogenated prior to reaction step (b).

For example, if R₁ is hydrogen and R₂ is as defined for formula I, this reaction can be shown by the following scheme:

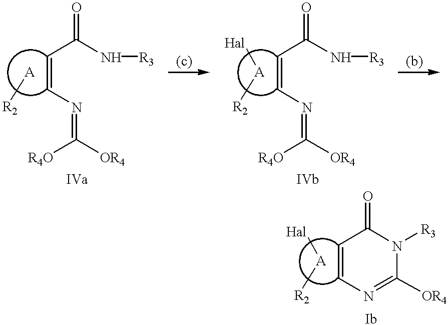

Particularly preferred is the chlorination of compounds of formula formula IV, in which A is thieno and R₁ and R₂ are both hydrogen.

Methods for halogenation [step (c)] are described e.g. in WO 97/33890 and include iodiniation with I₂, bromination with NBS (N-Bromsuccinimide) or Br₂, chlorination with NCS (N-Chlorsuccinimide) or Cl₂ or SO₂Cl₂, fluorination with FCl or other F⁺ reagents, in solvents as halogenated hydrocarbons, typically chlorobenzene, bromobenzene, chloroform, dichloromethane, trichloromethane, dichloroethane or trichloroethane; ethers, typically diethyl ether, tert-butylmethyl ether, glyme, diglyme, tetrahydrofuran or dioxane, as well as nitrogen containing compounds like triethylamine, piperidine, pyridine, alkylated pyridine, quinoline and isoquinoline.

Particularly preferred is the chlorination with NCS (N-Chlorsuccinimide), Cl₂ or SO₂Cl₂.

The general terms used hereinabove and hereinbelow have the following meanings, unless otherwise defined:

5-Membered heterocyclic rings A are for example thienyl, furanyl, oxazolyl, isoxazolyl, thiazolyl, pyrazolyl, imidazolyl, isothiazolyl and the corresponding partially or completely hydrogenated rings.

Alkyl groups are, in accordance with the number of carbon atoms, straight-chain or branched and will typically be methyl, ethyl, n-propyl, isopropyl, n-butyl, sec-butyl, isobutyl, tert-butyl, n-amyl, tert-amyl, 1-hexyl or 3-hexyl.

Alkenyl is straight-chain or branched alkenyl such as allyl, methallyl, 1-methylvinyl or but-2-en-1-yl. Preferred alkenyl radicals contain 3 to 4 carbon atoms in the chain.

Alkynyl can be straight-chain or branched and is typically propargyl, but-1-yn-1-yl or but-1-yn-3-yl; preferred is propargyl.

Halogen and halo substituents are fluoro, chloro, bromo or iodo. Fluoro, chloro and bromo are preferred.

Haloalkyl can contain identical or different halogen atoms, typically fluoromethyl, difluoro-methyl, difluorochloromethyl, trifluoromethyl, chloromethyl, dichloromethyl, trichloromethyl, 2,2,2-trifluoroethyl, 2-fluoroethyl, 2-chloroethyl, 2,2,2-trichloroethyl, 3,3,3-trifluoropropyl.

Alkoxy is typically methoxy, ethoxy, propyloxy, isopropyloxy, n-butyloxy, isobutyloxy, sec-butyloxy and tert-butyloxy. Methoxy and ethoxy are preferred.

Haloalkoxy is typically difluoromethoxy, trifluoromethoxy, 2,2,2-trifluoroethoxy, 1,1,2,2-tetrafluoroethoxy, 2-fluoroethoxy, 2-chloroethoxy and 2,2-difluoroethoxy.

Cycloalkyl is cyclopropyl, cyclobutyl, cyclopentyl, cyclohexyl or cycloheptyl.

Alkanoyl is either straight-chain or branched. Typical examples are formyl, acetyl, propionyl, butyryl, or pivaloyl.

Aryl is phenyl, benzyl or naphthyl; phenyl or benzyl are preferred.

R₁ and R₂ groups which are inert to the reactions are for example independently of the other hydrogen, halogen, C₁-C₄alkyl, C₁-C₄haloalkyl, C₂-C₄alkenyl, C₂-C₄haloalkenyl, C₂-C₄alkynyl, C₂-C₄haloalkynyl, Si—(C₁-C₆alkyl)₃, COO—C₁-C₄alkyl, COO—aryl, COOH, CH═N—C₁-C₄alkyl, C(CH₃)═N—C₁-C₄alkyl, SO—C₁-C₄alkyl, SO₂-C₁-C₄alkyl, OR₅, SR₆, NR₇R₈ or COR₉;

R₅ and R₆ are each independently of the other C₁-C₆alkyl, C₂-C₆alkenyl, C₂-C₆alkynyl or C₃-C₆cycloalkyl, each of which is unsubstituted or substituted by halogen, O—C₁-C₄alkyl, O—C₁-C₄haloalkyl, C₁-C₄alkoxy, S—C₁-C₄alkyl, SO—C₁-C₄alkyl, SO₂-C₁-C₄alkyl, CO—C₁-C₄alkyl, N═C₁-C₄alkyl, NH—C₁-C₄alkyl, N(C₁-C₄alkyl)₂, COO—C₁-C₄alkyl, COO—aryl, cyano, nitro, Si—(C₁-C₄alkyl)₃, phenyl, halophenyl, phenoxyphenyl, halophenoxyphenyl or naphthyl;

R₇ and R₈ are each independently of the other C₁-C₆alkyl, C₂-C₆alkenyl, C₂-C₆alkynyl or C₃-C₆cycloalkyl, each of which is unsubstituted or substituted by halogen, O—C₁-C₄alkyl, O—C₁-C₄haloalkyl, C₁-C₄alkoxy, S—C₁-C₄alkyl, SO—C₁-C₄alkyl, SO₂-C₁-C₄alkyl, CO—C₁-C₄-alkyl, N═C₁-C₄alkyl, NH—C₁-C₄alkyl, N(C₁-C₄alkyl)₂, COO—C₁-C₄alkyl, COO—aryl, cyano, nitro, Si—(C₁-C₄alkyl)₃, phenyl, halophenyl, phenoxyphenyl, halophenoxyphenyl or naphthyl;

R₉=hydrogen, C₁-C₄alkyl, optionally substituted phenyl or optionally substituted benzyl.

Preferred compounds of formula I which may be prepared by the process according to the invention are those, wherein

a) A is benzo, thieno, pyrido or pyridazino; or

b) R₁ and R₂ are independently hydrogen, halogen or halo-C₁-C₄alkyl; in particular those, wherein not both R₁ and R₂ are simultaneously hydrogen; or

c) R₃ and R₄ are independently C₁-C₆alkyl, C₂-C₆alkenyl, C₂-C₆alkynyl, C₃-C₆cycloalkyl, C₃-C₆cycloalkyl-C₁-C₆alkyl.

The method of this invention is particularly preferred for the preparation of compounds of formulae

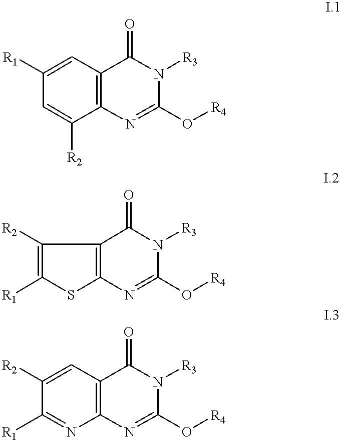

wherein

R₁ and R₂ are independently hydrogen, halogen or CF₃;

R₃ and R₄ are independently C₁-C₅alkyl or cyclopropylmethyl.

Preferred orthocarbonates of formula III are tetra-C₁-C₄alkyl orthocarbonates, as tetrabutyl orthocarbonate, tetrapropyl orthocarbonate and tetraethyl orthocarbonate.

The invention relates further to new intermediates of formula IV

[chemical structure]

wherein A, R₁, R₂, R₃ and R₄ are as defined for formula I.

Preferred intermediates are those of formulae

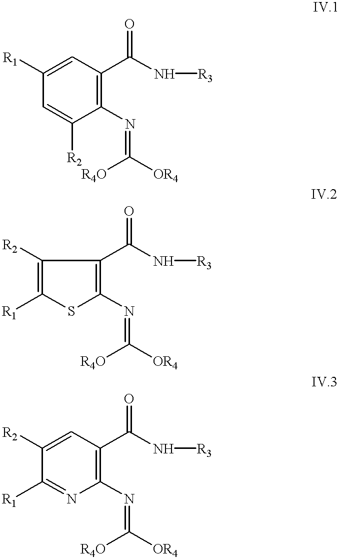

wherein

R₁ and R₂ are independently hydrogen, halogen or CF₃;

R₃ and R₄ are independently C₁-C₅alkyl or cyclopropylmethyl.

The invention relates further to compounds of formula II.2

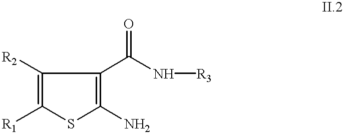

wherein R₁, R₂ and R₃ are as defined for formula I.

Preferred are compounds of formula II.2 wherein

a) R₁ and R₂ are independently hydrogen, halogen or halo-C₁-C₄alkyl; most preferably hydrogen; or

b) R₃ is C₁-C₈alkyl, C₂-C₆alkenyl, C₂-C₆alkynyl, C₃-C₆cycloalkyl, C₃-C₆cycloalkyl-C₁-C₆alkyl.

Particularly preferred are those wherein

R₁ and R₂ are independently hydrogen, halogen or CF₃, most preferably hydrogen; and R₃ is C₁-C₈alkyl.

Compounds of formula II.2 may be prepared by amidation of the corresponding acids or esters of formula

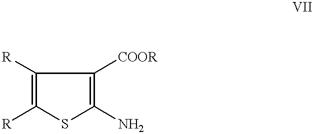

with R₃-NH₂, wherein R₁, R₂, and R₃ are as defined for formula II. Compounds of formula VII are known from the references indicated above.

The invention relates further to a process for the preparation of the compounds of formula II.2 according to the following reaction scheme:

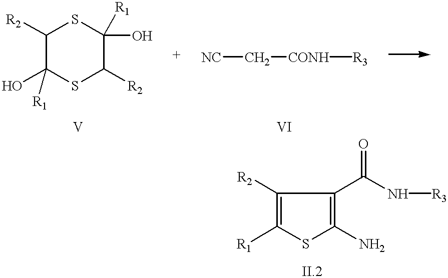

wherein R₁, R₂, and R₃ are as defined for formula II.2. Compounds of formula V and VI are known or can be prepared by known methods.

The reaction is carried out with or without a solvent; the temperature is not critical and may vary from 20° to 200° C.; preferably is a temperature of 80° to 170° C., most preferably at or near the boiling temperature of the solvent.

The reaction is advantageously carried out in the presence of a base, preferably in about equimolar amounts.

Suitable solvents and bases are those described above.

### PREPARATION EXAMPLES

In the following Examples, AcOEt means ethyl acetate.

### Example P-1: 6-Chloro-2-propoxy-3-propylquinazolin-4-one

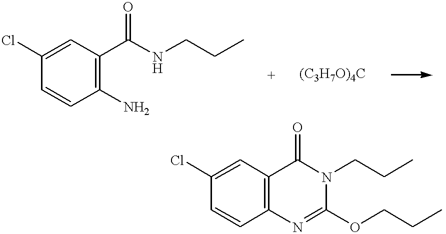

In a round bottom flask, a mixture of 1.0 g 2-amino-5-chlorobenzoic acid propylamide and 1.86 g tetrapropyl orthocarbonate is heated at 130° C. for 18 h. After cooling, the crude product is purified by column chromatography (eluant: hexane/AcOEt 6:1). Yield: 0.3 g 6-chloro-2-propoxy-3-propylquinazolin-4-one; m.p. 64-66° C.

### Example P-2: 2-(1,1-Dipropoxymethyleneamino)thiophene-3-carboxylic acid-propylamide

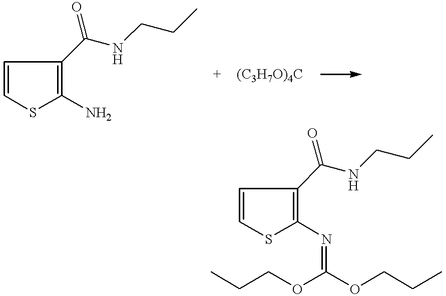

In a small destillation apparatus, a mixture of 1.0 g 2-aminothiophene-3-carboxylic acid propylamide and 2.0 g of tetrapropyl orthocarbonate is heated for 1 h at 130° C. and 2 h at 155° C. n-PrOH, which arises during the reaction is directly distilled out of the reaction flask. After cooling, the crude product is purified by column chromatography (eluant:hexane/AcOEt 1:2). Yield: 1.0 g of pure 2-(1,1 -dipropoxymethyleneamino)thiophene-3-carboxylic acid propylamide; m.p. 57-58° C.

### Example P-3: 5Chloro-2-(1,1-dipropoxymethylenamino)thiophene-3-carboxytic acid propylamide

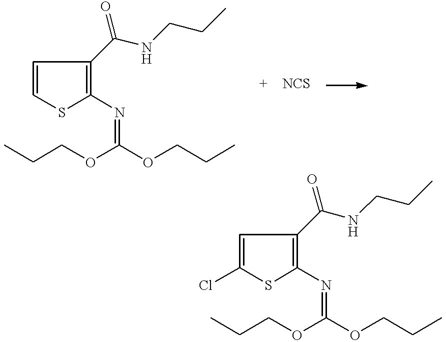

In a sulfonation flask 1.0 g 2-(1,1-dipropoxymethyleneamino)thiophene-3-carboxylic acid propylamide, are added, with stirring, to 20 ml absolute pyridine. The internal temperature is then raised to 60° C. and 0.5 g of N-chlorosuccinimide (NCS) are added in two portions. After stirring for 1 h at 60° C., the pyridine is removed in a water jet vacuum. The residue is taken up in EtOAc and the organic phase is washed twice with water. After drying of the organic phase, the solvent is removed in a water jet vacuum and the raw material purified by column chromatography over silica gel (eluant: hexane/AcOEt 1:1). Yield: 0.7 g 5-chloro-2-(1,1-dipropoxymethyleneamino)thiophene-3-carboxylic acid propylamide in the form of violet crystals; m.p. 88-90° C.

### Example P-4: 2-Propoxy-3-propylthieno[2.3d]pyrimidin-4-one

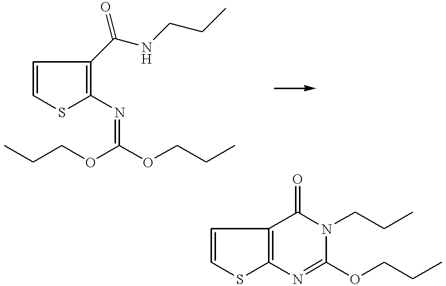

a) Method 1 (with sodium hydride):

In a sulfonation flask, 1.0 g of 2-(1,1-dipropoxymethyleneamino)thiophene-3-carboxylic acid propylamide is dissolved in 20 ml of absolute THF and 0.15 g of a ca. 55% NaH dispersion is added in small portions. The mixture is stirred for 15 minutes at room temperature and 1 h at reflux temperature. Then the solvent is removed in a water jet vacuum and the residue taken up in AcOEt. The organic phase is washed twice with water and after drying of the organic phase with Na₂SO₄, the solvent is removed in a water jet vacuum. The resulting crude product (yield: 0.8 g of 2-propoxy-3-propylthieno[2.3-d]-pyrimidine-4-one in the form of a brown liquid) can be used without further purification for the halogenation step.

b) Method 2 (with potassium carbonate):

In a sulfonation flask, 7.5 g of 2-(1,1-dipropoxymethyleneamino)thiophene-3-carboxylic acid propylamide is dissolved in 20 ml of absolute DMF and 6.2 g powdered potassium carbonate is added in one portion at room temperature. The mixture is stirred for 4 hours at 75-80° C. After cooling, the mixture is diluted with water and the water phase extracted three times with AcOEt. After drying the organic phase over sodium sulfate, the solvent is removed in a water jet vacuum and the crude material purified by column chromatography over silica gel (eluant: hexane/AcOEt 1:3). Yield: 5.8 g 2-propoxy-3-propylthieno[2.3-d]-pyrimidin-4-one in the form of slightly brown crystalls; m.p. 53-55° C.

### Example P-5: 6-Chloro-2-propoxy-3-propylthieno[2.3-d]pyrimidine-4-one

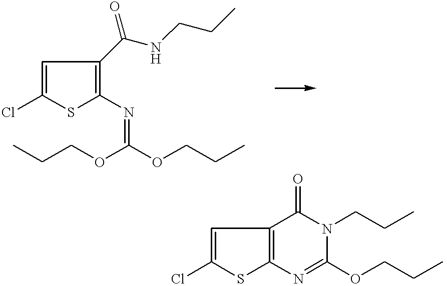

a) Method 1 (with sodium hydride):

In a sulfonation flask, 1.09 g of 5-Chloro-2-(1,1 -dipropoxymethyleneamino)-thiophene-3-carboxylic acid propylamide is dissolved in 20 ml of absolute THF and 0.08 g of a ca. 55% NaH dispersion is added in one portion. The mixture is stirred for 15 minutes at room temperature and 1 h at reflux temperature. Then the solvent is removed in a water jet vacuum and the residue taken up in AcOEt. The organic phase is washed twice with water and after drying of the organic phase with Na₂SO₄, the solvent is removed in a water jet vacuum. The resulting crude product is purified by column chromatography over silica gel (eluant: hexane/AcOEt 5:1). Yield: 0.8 g 6-chloro-2-propoxy-3-propylthieno[2.3-d]pyrimidin-4-one in the form of a yellowish powder; m.p.: 63-65°.

b) Method 2 (with potassium carbonate):

In a sulfonation flask, 3.5 g of 5-chloro-2-(1,1 -dipropoxymethyleneamino)thiophene-3-carboxylic acid propylamide is dissolved in 30 ml of absolute DMF and 1.93 g powdered potassium carbonate is added in one portion at room temperature. After stirring for 4 hours at ca 75° C., water is added to the precooled reaction mixture and the water phase extracted three times with AcOEt. Then work up is continued as described in example P-4 (method 2). Yield: 2.4 g of 6-chloro-2-propoxy-3-propylthieno[2.3-d]pyrimidin-4-one in the form of slightly brown crystalls, m.p.: 63-67°

### Example P-6: 3-Pemtyl-2-propoxythieno[2.3-d]pyrimidin-4-one

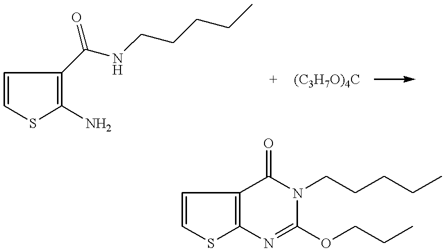

In a small destination apparatus, a mixture of 5 g 2-aminothiophene-3-carboxylic acid pentyl-amide and 8.2 g tetrapropylorthocarbonate is heated for 15 hours at 150-160° C. n-PrOH, which arises during the reaction is directly distilled out of the reaction flask. Then excess tetrapropylorthocarbonate is distilled of in vacuo and the residue purified by column chromatography over silica gel (eluant: hexane/AcOEt 2:1). Yield: 3.5 g 3-pentyl-2-propoxythieno[2.3-d]pyrimidin-4-one in the form of a brown oil.

### Example P-7: 2-Amimothiophene-3-carboxylic acid propylamid

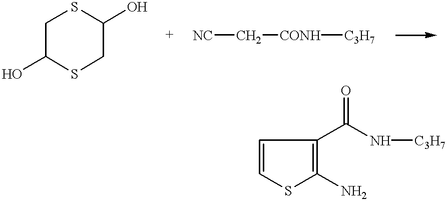

In a sulfonation flask, to a mixture of 2.53 g 2,5-dihydroxy-1,4-dithiane and 4.2 g cyanoacetic acid propylamide in 20 ml of MeOH, 1.3 ml of triethyl amine are added dropwise at a constant temperature of ca. 40° C. Then the mixture is heated at reflux temperature for 2 hours. After cooling 100 ml of ice water are added dropwise. The resulting precipitate (the product) is filtered off and purified by solving in CHCl₃, heating in the presence of charcoal and hot filtration. The CHCl₃ is removed in a water jet vacuum. Yield: 3.15 g brownish oil, which is is pure enough for further transformations. After several days at room temperature the oil begins to crystalize, m.p. of the crystals 140-142° C.

In analogous manner, the compounds of WO 97/48684, WO 97/33890, WO 97/02262 and WO 94/26722 may be prepared, as well as the new intermediate compounds of the Tables 1-3.

TABLE 1
Compounds of the formula

| | |
|---|---|
| 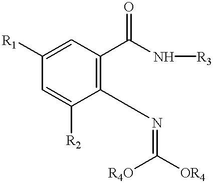 | IV.1 |

wherein R₁, R₂ R₃ and R₄ have the meaning given in Table A

TABLE 2
Compounds of the formula

| | |
|---|---|
| 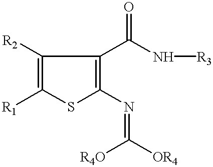 | IV.2 |

wherein R₁, R₂ R₃ and R₄ have the meaning given in Table A

TABLE 3
Compounds of the formula

| | |
|---|---|
| 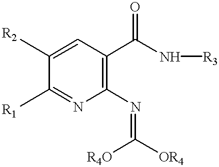 | IV.3 |

wherein R₁, R₂ R₃ and R₄ have the meaning given in Table A

TABLE A
Me: methyl; Et: ethyl

| | | | | | Formula: |
|---|---|---|---|---|---|
| | | | | | phys. data |
| No. | R₁ | R₂ | R₃ | R₄ | (melting point) |
| 1 | Cl | H | Me | Et | |
| 2 | Br | H | Et | Et | |
| 3 | H | H | n-propyl | n-propyl | IV.2: 57-58° C. |
| 4 | Cl | H | n-propyl | n-propyl | IV.2: 88-90° C. |
| 5 | Br | H | n-propyl | n-propyl | |
| 6 | I | H | n-propyl | n-propyl | |
| 7 | Br | H | n-propyl | i-propyl | |
| 8 | I | H | n-propyl | i-propyl | |
| 9 | Cl | H | n-propyl | n-butyl | |
| 10 | Br | H | n-propyl | n-butyl | |
| 11 | Br | H | n-propyl | i-butyl | |
| 12 | Cl | H | n-butyl | n-propyl | IV.2: 73-74° C. |
| 13 | Br | H | n-butyl | n-propyl | |
| 14 | Br | H | n-butyl | i-propyl | |
| 15 | I | H | n-butyl | i-propyl | |
| 16 | Br | H | n-butyl | n-butyl | |
| 17 | I | H | n-butyl | n-butyl | |
| 18 | Cl | H | i-butyl | n-propyl | |
| 19 | Br | H | i-butyl | n-propyl | |
| 20 | Br | H | i-butyl | i-propyl | |
| 21 | I | H | i-butyl | i-propyl | |
| 22 | Br | H | CH₂-cyclopropyl | n-propyl | |
| 23 | Br | H | CH₂-cyclopropyl | i-propyl | |
| 24 | Br | H | CH₂-cyolopropyl | n-butyl | |
| 25 | Br | H | n-propyl | CH₂-cyclopropyl | |
| 26 | Br | H | n-butyl | CH₂-cyclopropyl | |
| 27 | Cl | Cl | Et | Et | |
| 28 | Br | Br | Et | Et | |
| 29 | Br | Br | Et | n-propyl | |
| 30 | I | I | Et | n-propyl | |
| 31 | Cl | Cl | n-propyl | n-propyl | |
| 32 | Br | Br | n-propyl | n-propyl | |
| 33 | I | I | n-propyl | n-propyl | |
| 34 | Cl | Cl | n-propyl | i-propyl | |
| 35 | Br | Br | n-propyl | i-propyl | |
| 36 | I | I | n-propyl | i-propyl | |
| 37 | Br | Br | n-propyl | n-butyl | |
| 38 | I | I | n-propyl | n-butyl | |
| 39 | Br | Br | n-propyl | i-butyl | |
| 40 | I | I | n-propyl | i-butyl | |
| 41 | Br | Br | n-butyl | n-propyl | |
| 42 | I | I | n-butyl | n-propyl | |
| 43 | Br | Br | i-butyl | n-propyl | |
| 44 | Br | Br | i-butyl | i-propyl | |
| 45 | Br | Br | CH₂-cyclopropyl | n-propyl | |
| 46 | I | I | CH₂-cyclopropyl | n-propyl | |
| 47 | Br | Br | n-propyl | CH₂-cylcopropyl | |
| 48 | Br | Br | n-propyl | n-pentyl | |
| 49 | Cl | Cl | n-propyl | n-pentyl | |
| 50 | Br | Br | n-propyl | allyl | |
| 51 | I | I | n-propyl | allyl | |
| 52 | Br | Br | n-propyl | propargyl | |
| 53 | H | H | n-butyl | n-propyl | IV.2: 48-50° C. |

TABLE 4

| | |
|---|---|
| Compounds of formula II.2 | II.2 |
| 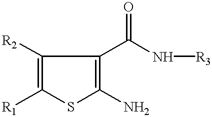 | |

| | | | | phys. data |
|---|---|---|---|---|
| No. | R₁ | R₂ | R₃ | (melting point) |
| 4.1 | H | H | Me | 145-147° C. |
| 4.2 | Br | H | Et | |
| 4.3 | H | H | n-propyl | 140-142° C. |
| 4.4 | Cl | H | n-propyl | |
| 4.5 | Br | H | n-propyl | |
| 4.6 | I | H | n-propyl | |
| 4.7 | Cl | Cl | n-propyl | |
| 4.8 | H | Cl | n-propyl | |
| 4.9 | H | H | n-butyl | 92-94° C. |
| 4.10 | Cl | H | n-butyl | |
| 4.11 | Br | H | n-butyl | |
| 4.12 | I | H | n-butyl | |
| 4.13 | Cl | H | i-butyl | |
| 4.14 | Br | H | i-butyl | |
| 4.15 | Br | H | CH₂-cyolopropyl | |
| 4.16 | H | H | n-pentyl | 78-80° C. |
| 4.17 | H | H | n-hexyl | |
| 4.18 | H | H | n-heptyl | |
| 4.19 | H | H | n-ootyl | |
| 4.20 | H | H | OMe | 144-147° C. |
| 4.21 | H | H | OEt | |

1. A process for the preparation of a compound of the formula I 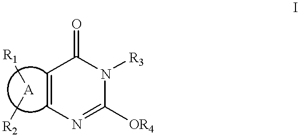
wherein
A is a fused 5-membered heterocyclic ring which may be saturated or unsaturated, aromatic or non-aromatic and which may contain one or two hetero atoms 0, S and/or N, or is fused benzo, pyrido or pyridazino;
R₁ and R₂ are groups which are inert to the reactions;
R₃ is C₁-C₈alkyl, C₂-C₈alkenyl, C₂-C₈alkynyl, C₃-C₆cycloalkyl or C₃-C₆cycloalkyl-C₁-C₆alkyl, each of which is unsubstituted or substituted by halogen; or is O—C₁-C₄alkyl, O—C₁-C₄haloalkyl, C₁-C₄alkoxy, S—C₁-C₄alkyl, SO—C₁-C₄alkyl, SO₂-C₁-C₄alkyl, CO—C₁C₄alkyl, N═CH—C₁-C₄alkyl, N═C(C₁-C₄alkyl)₂, NH—C₁-C₄alkyl, N(C₁-C₄alkyl)₂, COO—C₁-C₄alkyl, COO-aryl, cyano, nitro, Si—(C₁-C₄alkyl)₃, phenyl, halophenyl, phenoxyphenyl, halophenoxyphenyl or naphthyl; and R₄ is C₁-C₆alkyl, C₂-C₆alkenyl, C₂-C₆alkynyl, C₃-C₆cycloalkyl or C₃-C₆cycloalkyl-C₁-C₆alkyl, each of which is unsubstituted or substituted by halogen; or is O—C₁-C₄alkyl, O—C₁-C₄haloalkyl, C₁-C₄alkoxy, S—C₁-C₄alkyl, SO—C₁-C₄alkyl, SO₂-C₁-C₄alkyl, CO—C₁-C₄alkyl, N═CH—C₁-C₄alkyl, N═C(C₁-C₄alkyl)₂, NH—C₁-C₄alkyl, N(C₁-C₄alkyl)₂, COO—C₁-C₄alkyl, COO-aryl, cyano, nitro, Si—(C₁-C₄alkyl)₃, phenyl, halophenyl, phenoxyphenyl, halophenoxyphenyl or naphthyl; in which process
(a) a compound of the formula II, wherein A, R₁, R₂ and R₃ are as defined for formula I, is reacted with an orthocarbonate of the formula III, wherein R₄ is as defined for formula I and Y is OR₄, CN or NO₂, to give the intermediate compound of formula IV; and subsequently
(b) the compound of the formula IV is cyclized to a compound of the formula I

[chemical structure]

2. A process according to claim 1, wherein reaction step (a) is carried out in the presence of an acid and in the absence of water.

3. A process according to claim 1, wherein reaction step (b) is carried out in the presence of a base.

4. A process according to claim 1, wherein in formula I R₁ and/or R₂ are halogen, in which process a compound of the formula IV, in which R₁ and/or R₂ are hydrogen, is halogenated prior to reaction step (b).

5. A process according to claim 1, wherein the compound of the formula IV is not isolated.

6. A process according to claim 1, wherein in the compounds of the formulae I to IV
A is benzo, thieno, pyrido or pyridazino;
R₁ and R₂ are independently hydrogen, halogen or halo-C₁-C₄alkyl;
R₃ and R₄ are independently C₁-C₆alkyl, C₂-C₆alkenyl, C₂-C₆alkynyl, C₃-C₆cycloalkyl, C₃-C₆cycloalkyl-Cl-C₆alkyl;

7. A process according to claim 6 for the preparation of a compound of the formula 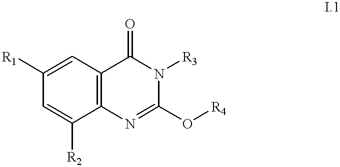
where
R₁ and R₂ are independent hydrogen, halogen or CF₃;
R₃ and R₄ are independently C₁-C₅alkyl or cyclopropylmethyl.

8. A process according to claim 6 for the preparation of a compound of the formula 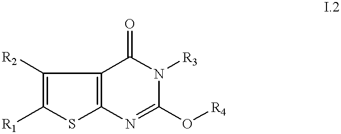
wherein
R₁ and R₂ are independently hydrogen, halogen or CF₃;
R₃ and R₄ are independently C₁-C₅alkyl or cyclopropylmethyl.

9. A process according to claim 6 for the preparation of a compound of the formula 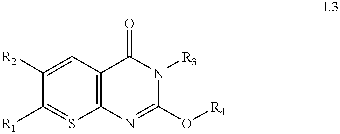
wherein
R₁ and R₂ are independently hydrogen, halogen or CF₃;
R₃ and R₄ are independently C₁-C₅alkyl or cyclopropylmethyl.

10. A compound of the formula 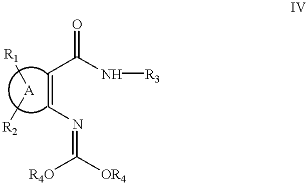
where A,R₁,R₂,R₃, and R₄ are as defined for formula I.

11. A compound of the formula II.2 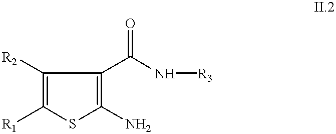
wherein R₁, R₂ and R₃ are as defined for formula I.

12. A process for the preparation of a compound of formula II.2, wherein a compound of formula V is reacted with a compound of formula VI [chemical structure]
